(19) United States
(12) Patent Application Publication
(10) Pub. No.: US 2026/0151735 A1
(43) Pub. Date: Jun. 4, 2026

(54) MONITORING AND OPTIMIZING WATER PRE-TREATMENT BY ULTRAVIOLET ILLUMINATION AND ADDITION OF CHEMICAL AGENTS

(71) Applicant: Atlantium Technologies Ltd, Beit-Shemesh (IL)

(72) Inventors: Yariv ABRAMOVICH, Kfar Gibton (IL); Ytzhak ROZENBERG, Caesarea (IL); Amichai FELDER, Givatayim (IL); Rachel GOTTLIEB, Jerusalem (IL); Assaf LOWENTHAL, Rehovot (IL)

(73) Assignee: Atlantium Technologies Ltd, Beit-Shemesh (IL)

(21) Appl. No.: 19/119,772

(22) PCT Filed: Oct. 18, 2023
(86) PCT No.: PCT/IL2023/051085

§ 371 Date: Apr. 10, 2025

Related U.S. Application Data
(60) Provisional application No. 63/417,018, filed on Oct. 18, 2022.
(60) Provisional application No. 63/419,041, filed on Oct. 25, 2022.

(51) Int. Cl. B01D 61/04; B01D 61/02; B01D 61/12; B01D 65/10; C02F 1/00; C02F 1/32; C02F 1/44; C02F 5/00; C02F 9/00; G01N 15/08; G01N 33/18

(52) U.S. Cl. CPC B01D 61/04; B01D 61/025; B01D 61/12; B01D 65/109; C02F 9/00; G01N 15/0826; G01N 33/18; B01D 2311/04; C02F 1/004; C02F 1/325; C02F 1/441; C02F 5/00; C02F 2201/326; C02F 2209/02; C02F 2209/03; C02F 2303/04; C02F 2303/20; G01N 2015/084

(57) ABSTRACT

Water treatment systems and methods are provided for monitoring and optimizing pre-treatment of water delivered to a water treatment sub-system. Monitoring modules and methods are provided, which receive measurements of pressure differences over filtration units, determine the type and extent of degradation of membranes and apply ultraviolet (UV) radiation if biological fouling is detected, to reduce application of chemicals. Purification systems and methods are provided for purifying solutions of lactic acid and/or lactate by UV radiation, to improve the quality of poly lactic acid (PLA) produced therefrom. The color of the solution is monitored to tune the wavelength and intensity of the applied UV.

### BACKGROUND OF THE INVENTION

### 1. Technical Field

The present invention relates to the field of water pre-treatment, and more particularly, to optimizing UV and chemical pre-treatment.

Also, the present invention relates to the field of polylactic acid (PLA), and more particularly, to color removal from PLA using UV radiation.

### 2. Discussion of Related Art

Reverse osmosis (RO) facilities are used to desalinate saline or brackish water to yield potable water, by pressing the delivered water through fine sets of membranes to remove their salt content. The RO process is sensitive to membrane degradation due to biological fouling and mineral scaling, which constitute the accumulation of respective material clogging the membranes. Due to these processes, delivered water are pre-treated, and the membranes are cleaned regularly.

Lactic acid is an organic acid that is used in multiple applications such as food and beverages, homecare, personal care and in industrial products such as sanitation and descaling. Lactic acid is also a building block to produce PLA (polylactic acid) bioplastic. PLA has become a popular material due to it being economically produced from renewable resources. PLA is also the most widely used plastic filament material in 3D printing. Its low melting point, high strength, low thermal expansion, good layer adhesion, and high heat resistance when annealed make it an ideal material for this purpose. Lactic acid can be made from organic waste using scalable biological processes.

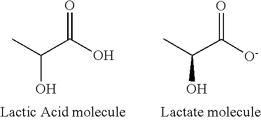

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a water treatment system for pre-treating water delivered to a water treatment sub-system, the water treatment system comprising: a ultraviolet (UV) treatment module configured to treat the delivered water with UV, a chemical treatment module comprising a chemical addition unit configured to add chemicals to the delivered water and a filtration unit configured to filter solids from the delivered water, and a monitoring module configured to: receive measurements comprising at least a pressure difference over the filtration unit and/or a pressure difference over the water treatment sub-system, determine, based on the measurements, a type and extent of a degradation of membranes of the water treatment sub-system due to biological fouling and/or mineral scaling, and adjust, based on the determined type and extent of the degradation, a UV dose to treat biological fouling and/or the addition of chemicals to treat mineral scaling—to maximize a performance of the water treatment sub-system.

One aspect of the present invention provides a monitoring module of a water treatment system for pre-treating water delivered to a water treatment sub-system, the monitoring module configured to: receive measurements comprising at least a pressure difference over a filtration unit of the water treatment system and/or a pressure difference over the water treatment sub-system, determine, based on the measurements, a type and extent of degradation of membranes of the water treatment sub-system due to biological fouling and/or mineral scaling, and adjust, based on the determined type and extent of the degradation, a UV dose applied by a UV treatment module to the delivered water to treat biological fouling and/or an addition of chemicals to the delivered water to treat mineral scaling—to maximize a performance of the water treatment sub-system.

One aspect of the present invention provides a method of managing water pre-treatment of delivered water to a water treatment sub-system, the method comprising: receiving measurements comprising at least a filtering pressure difference and/or a pressure difference over the water treatment sub-system, determining, based on the measurements, a type and extent of degradation of membranes of the water treatment sub-system due to biological fouling and/or mineral scaling, and adjusting, based on the determined type and extent of the degradation, a UV dose applied to the delivered water to treat biological fouling and/or addition of chemicals to the delivered water to treat mineral scaling—to maximize a performance of the water treatment sub-system.

One aspect of the present invention provides a water pre-treatment method comprising: treating, with UV, delivered water to a water treatment sub-system, adding chemicals and consecutively filtering the delivered water, receiving measurements comprising at least a filtering pressure difference and/or a pressure difference over membranes of the water treatment sub-system, determining, based on the measurements, a type and extent of degradation of membranes of the water treatment sub-system due to biological fouling and/or mineral scaling, and adjusting, based on the determined type and extent of the degradation, a UV dose applied to the delivered water to treat biological fouling and/or addition of chemicals to the delivered water to treat mineral scaling—to maximize a performance of the water treatment sub-system.

One aspect of the present invention provides a computer program product comprising a non-transitory computer readable storage medium having computer readable program embodied therewith, the computer readable program comprising: computer readable program configured to receive measurements comprising at least a filtering pressure difference and/or a pressure difference over the water treatment sub-system, computer readable program configured to determine, based on the measurements, a type and extent of degradation of membranes of the water treatment sub-system due to biological fouling and/or mineral scaling, and computer readable program configured to adjust, based on the determined type and extent of the degradation, a UV dose applied to the delivered water to treat biological fouling and/or addition of chemicals to the delivered water to treat mineral scaling—to maximize a performance of the water treatment sub-system.

One aspect of the present invention provides a purification system comprising at least one medium pressure ultraviolet (MPUV) lamp configured to irradiate a solution of lactic acid and/or lactate, at least one sensor for color detection of the irradiated solution, and a controller configured to control the at least one MPUV lamp with respect to measurements of the at least one sensor, to purify the solution of lactic acid and/or lactate according to a specified criterion.

One aspect of the present invention provides a purification method comprising irradiating a solution of lactic acid and/or lactate by ultraviolet radiation from at least one medium pressure ultraviolet (MPUV) lamp, detecting color of the irradiated solution, and controlling the at least one MPUV lamp with respect to the detected color, to purify the solution of lactic acid and/or lactate according to a specified criterion.

One aspect of the present invention provides a computer program product comprising a non-transitory computer readable storage medium having computer readable program embodied therewith, the computer readable program configured to control at least one medium pressure ultraviolet (MPUV) lamp irradiating a solution of lactic acid and/or lactate by ultraviolet radiation for purification, with respect to a detected color of the irradiated solution, according to a specified criterion.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout. In the accompanying drawings:

FIGS. 1A and 1B are high-level schematic block diagrams of a water treatment system for pre-treating water delivered to a water treatment sub-system, according to some embodiments of the invention.

FIG. 1C provides an example of water treatment by the water treatment system indicating the two types of pressure difference increase and the optimization of UV treatment to counter biofouling and reduce the required chemical treatment, according to some embodiments of the invention.

FIG. 2 is a high-level flowchart illustrating methods, according to some embodiments of the invention.

FIG. 3 is a high-level block diagram of exemplary processing unit(s), which may be used with embodiments of the present invention.

FIG. 4 is a high-level schematic block diagram of a purification system for purifying a solution of lactic acid and/or lactate received from a biological reactor, according to some embodiments of the invention.

FIG. 5 is a high-level flowchart illustrating purification methods, according to some embodiments of the invention.

FIGS. 6A-6D provide non-limiting examples for UV treatment of solutions, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing”, “deriving” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Embodiments of the present invention provide efficient and economical methods and mechanisms for optimizing the application of water pre-treatment means and thereby provide improvements to the technological field of water pre-treatment. Water treatment systems and methods are provided for monitoring and optimizing the pre-treatment of water delivered to a water treatment sub-system. Ultraviolet (UV) is used to remove biological fouling from the delivered water and addition of chemicals and filtering are used to remove mineral scaling from the delivered water. Monitoring modules and methods are provided, which receive measurements of pressure differences over the filtration unit and/or the water treatment sub-system, and optionally additional system parameters, determine, based on the measurements, a type and extent of degradation of membranes of the water treatment sub-system-due to biological fouling and/or mineral scaling, and adjust, based on the determined type and extent of the degradation, the UV dose applied to the delivered water to treat biological fouling and/or addition of chemicals to the delivered water to treat mineral scaling—to increase the productivity and reduce maintenance of the water treatment sub-system by optimizing UV vs. chemical pre-treatment. Advantageously, disclosed optimized pre-treatment increases the productivity of alternative water sources such as produced, e.g., by RO water desalination, while simultaneously reducing the use of chemicals that are potentially harmful to the environments, thus reducing the environmental footprint of the desalination facility. Similarly, optimizing water pre-treatment as disclosed herein may be applied to a wide range of water treatment sub-systems, which includes, in addition to RO systems, systems for water polishing such as Mixed Bed resin (MB) systems, Ion Exchanger (IX) systems, Electrodionization (EDI) systems, as well as reducing damage to the filtration units themselves and to piping and water distributions systems throughout the water pre-treatment and treatment systems. Disclosed embodiments enable optimal protection of expensive water treatment equipment that is prone to membrane degradation, by optimally pre-treating the water to prevent biological fouling and mineral scaling implementing minimal interventions.

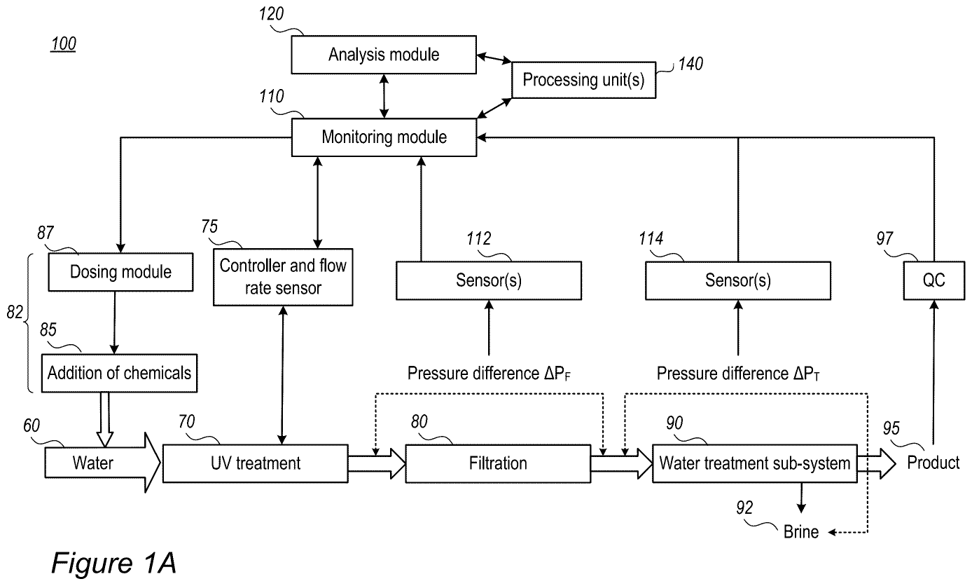

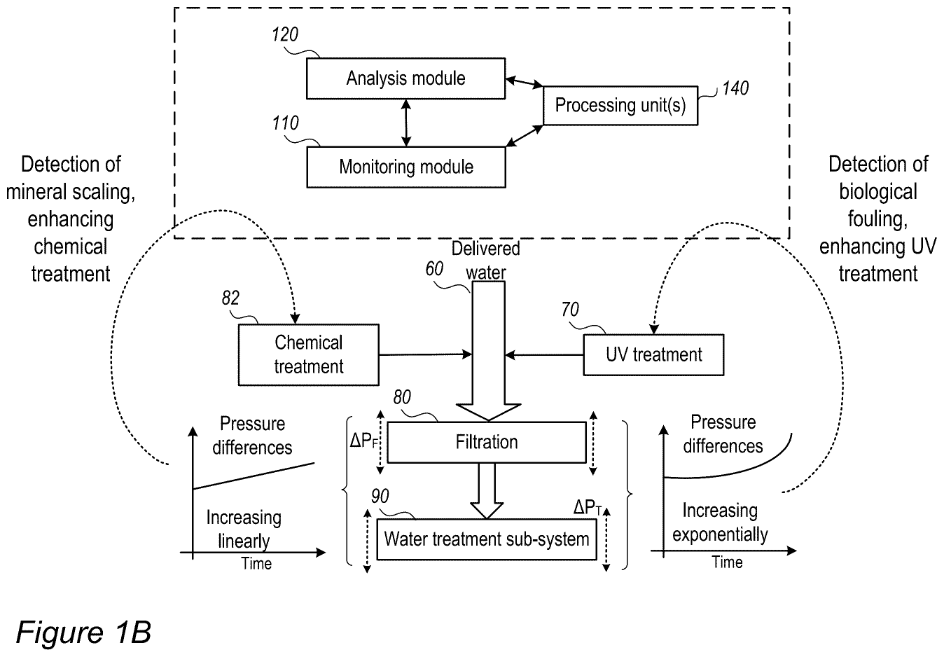

FIGS. 1A and 1B are high-level schematic block diagrams of a water treatment system 100 for pre-treating water 60 delivered to a water treatment sub-system 90, according to some embodiments of the invention. As illustrated schematically in FIG. 1A, water treatment system 100 comprises a ultraviolet (UV) treatment module 70 configured to treat the delivered water with UV, a chemical treatment module 82 comprising a chemical addition unit 85 configured to add chemicals to the delivered water (e.g., upon controlling by a dosing module 85) and a filtration unit 80 configured to filter solids from the delivered water. Water treatment system 100 further comprises a monitoring module 110 configured to receive measurements comprising at least a pressure difference over filtration unit 80 (denoted ΔP_{F}) received, e.g., from sensor(s) 112 and/or a pressure difference over water treatment sub-system 90 (denoted APT), e.g., from sensor(s) 114, determine, based on the measurements, a type and extent of degradation of membranes of water treatment sub-system 90 (e.g., a RO system)—due to biological fouling and/or mineral scaling, and adjust, based on the determined type and extent of the degradation, a UV dose to treat biological fouling and/or the addition of chemicals to treat mineral scaling)—to maximize the performance of water treatment sub-system 90 (e.g., the RO system). Additional measurements may include a flow rate through UV treatment module 70, received, e.g., from flow rate sensor(s) associate with controller(s) 75 of UV treatment module 70. Additional measurements may include quality control (QC) parameters 97 concerning product water 95 as well as parameters relating to brine 92 produced by water treatment sub-system 90 such as the throughput, pressure, temperature, quality (e.g., residual salinity or specific residual ions or compounds), etc. measured for product water 95 and/or for brine 92.

Certain embodiments comprise monitoring module 110 which may be configured as a management module for controlling, e.g., dosing module 87 and/or controller 75 to minimize membrane fouling/scaling in water treatment sub-system 90 and to maximize the performance of water treatment sub-system 90, e.g., by reducing the required maintenance operations and increasing the amount of produced water. Monitoring module 110 may comprise and/or be associated with an analysis module 120 for enhancing the optimization process, e.g., implementing artificial intelligence (AI) methods.

Various parameters may be used to monitor the membrane operation to determine if a developing degradation is mainly due to biological fouling or mineral scaling. The inventors have noted that biological fouling comprises the accumulation of biological organisms, and is typically characterized by an exponential growth of the fouling material, while mineral scaling comprises accumulation of inorganic material, and is typically characterized by linear growth of the scaling material. Accordingly, the inventors have found out that by monitoring the development of the pressure differential across the membranes (measured by sensor 114), as well as optionally by monitoring other parameters such as the filtration differential pressure (measured by sensor 112), water treatment throughput, the applied pressure, the water temperature and the product water quality—the type and cause of developing degradation may be evaluated, and a corresponding treatment method may be preferred.

As illustrated schematically in FIG. 1B, detection of a linear increase in the pressure difference across one or more membrane in the system, possibly associated with additional measurements, may be used to detect mineral scaling, and be treated by enhancing chemical treatment of the delivered water; while detection of an exponential increase in the pressure difference across one or more membrane in the system, possibly associated with additional measurements, may be used to detect biological fouling, and be treated by enhancing UV treatment of the delivered water.

Machine learning (ML) may be applied to derive the relations between the monitored parameters and the developing type of degradation (e.g., fouling or scaling), e.g., by analysis module 120.

For example, if biological fouling is detected, e.g., by monitoring module 110 detecting an exponential growth of the pressure difference across water treatment sub-system 90-UV treatment 70 may be applied or enhanced, and monitored to confirm reduction of the fouling. If mineral scaling is detected, e.g., by monitoring module 110 detecting a linear growth of the pressure difference across water treatment sub-system 90, chemical addition 85 may be applied or modified to reduce the scaling. In various embodiments, the type of applied treatment, e.g., adding chemicals and/or applying UV radiation, or modifying any of these options, may be considered with respect to the monitored type of membrane degradation, and adjusted accordingly.

Monitored pressures across water treatment sub-system 90 may include the inlet pressure, the pressure of product water 95, the pressure of brine 92 rejected by water treatment sub-system 90, and differences between these pressures. Additional monitored parameters may include parameters related to UV treatment 70 (e.g., UV transmittance, illumination intensity, etc.), chemical treatment module 82 (e.g., amounts and types of added chemicals) and filtration 80 (e.g., pressure difference, type and accumulation rate of filtered material, optionally with respect the type and amount of chemical additives), which analysis module 120 may relate over time to the optimized parameters of water treatment system 100 (e.g., productivity and/or efficiency), e.g., using ML and/or AI.

In non-limiting examples, monitored parameters of UV treatment 70 may include water transmittance to UV (termed UVT) measured by built-in sensor(s), e.g., associated with the UV lamps, which may be monitored in real-time. Illumination intensity of individual lamps may likewise be measured and monitored in real-time to provide additional parameters (e.g., detection of and compensation for reduced UV treatment efficiency, optimized with respect to maintenance operations). It is noted that the UV treatment efficiency under a certain flow rate depends on both the lamp output and the water transmittance, which combine to quantify the actual UV dose that is applied in the treatment. It is further noted that UVT may be affected by the addition of chemicals and the level of water contamination, so that UVT may be included in the monitoring of the overall water pre-treatment efficiency. Controller(s) and/or sensor(s) may be associated separately with each or several of the modules in UV treatment system 70 to provide more spatial and temporal details concerning the water flow and the real-time UV treatment parameters in order to maximize operational flexibility. It is noted that UV treatment systems 70 with lamps set perpendicular to the direction of water flow may enable continued operation even during lamp replacement, possibly with compensation via chemical treatment during the off-time of one or more of the UV lamps—controlled by monitoring module 110.

Monitoring module 110 may be configured to apply machine learning (e.g., via analysis module 120) to optimize treatment, e.g., maximize the overall production, minimize the overall application of chemicals or optimize these and other parameters—with respect to the monitored parameters and related characteristics of the fouling and/or scaling processes and the applied treatment to reduce the fouling and/or scaling—to achieve synergic combination of UV treatment and chemical treatment (respectively) in water treatment system 100.

Accordingly, in various embodiments, monitoring module 110 may be further configured to further receive measurements of at least one of: throughput, pressure, temperature and/or quality of product water and brine from water treatment sub-system 90, UV transmittance and/or illumination intensity of UV treatment module 70, and/or amounts and types of added chemicals by chemical treatment module 82, and monitoring module 110 may further comprise or be associated with analysis unit 120 configured to apply machine learning (ML) algorithms to analyze the measurements over time and further adjust the UV dose and the addition of chemicals to maximize the performance.

For example, in preliminary experiments the application of disclosed monitoring module and methods reduced by 50% the required number of maintenance interruptions (Clean In Place-CIP procedures, reduced from 14 to 7 within seven months) in a RO facility, while actually increasing water production by 5% (overall hourly permeate yield has risen from 69 to 73 m³/hr). In another RO facility, the application of disclosed monitoring module and methods reduced by 70% the required number of maintenance interruptions (reduced the numbers of CIPs from 13 to 4 within seven months). It is noted however, that these improvements were due to the addition of UV pre-treatment rather than to the optimization of UV vs. chemical pre-treatment. Still, as adding UV treatment has proved so effective, optimizing UV treatment with chemical pre-treatment is expected to further enhance the increase in performance.

FIG. 1C provides an example of water treatment by water treatment system 100 indicating the two types of pressure difference increase and the optimization of UV treatment to counter biofouling and reduce the required chemical treatment, according to some embodiments of the invention. The example is from system 100 pre-treating sea water for a desalination plant using RO membranes. The graph illustrates exponential increases in the pressure difference which indicate organic biofouling, activation of additional UV treatment, and linear increases in the pressure difference which indicate inorganic mineral scaling. Implementing UV treatment 70 in the disclosed way resulted in reduced and even stopped application of chlorine for chemical treatment 82, and subsequently chemical application of sodium bisulfite (SBS) to remove the chlorine to protect the RO membranes therefrom. FIG. 1C clearly illustrates that system 100 can be effectively used to detect the kind of water contaminant (biofouling and/or inorganic scaling) and treat the water correspondingly by UV or chemically, thus minimizing the required chemical and maximizing the treatment efficiency.

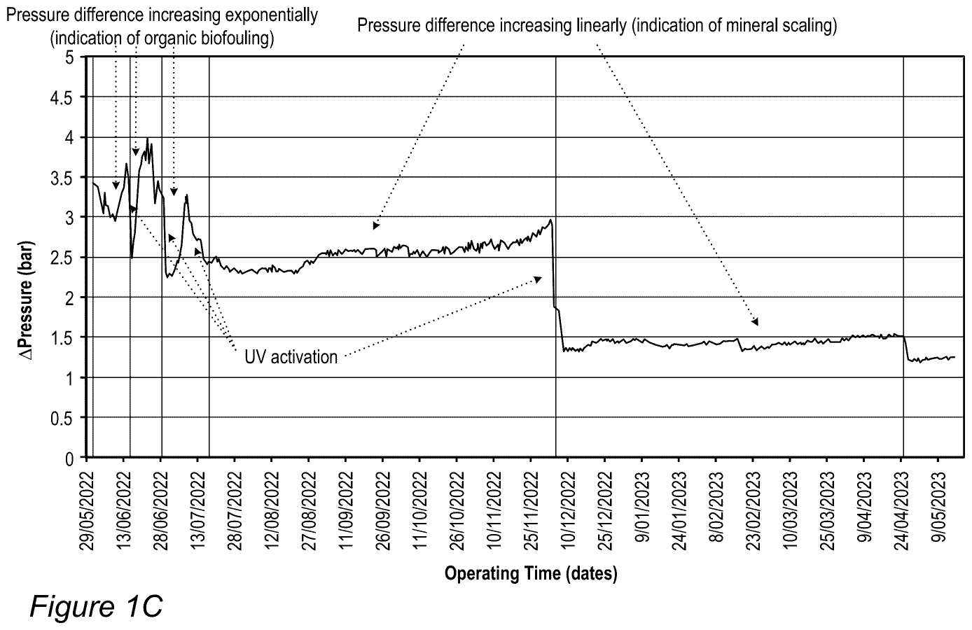

FIG. 2 is a high-level flowchart illustrating methods 200, according to some embodiments of the invention. The method stages may be carried out with respect to system 100 and/or monitoring module 110 described above, which may optionally be configured to implement methods 200. Methods 200 may be at least partially implemented by at least one computer processor, e.g., in monitoring module 110 and/or in analysis unit 120. Certain embodiments comprise computer program products comprising a computer readable storage medium having computer readable program embodied therewith and configured to carry out the relevant stages of methods 200. Methods 200 may comprise the following stages, irrespective of their order.

Methods 200 comprise managing water pre-treatment of delivered water to a water treatment sub-system (stage 202), such as a RO or any other membrane-based water treatment facility. The water pre-treatment process, which may be part of method 200, comprises pre-treating, with UV, delivered water to the water treatment sub-system (stage 205) and adding chemicals and consecutively filtering the delivered water (stage 207). Managing 202 comprises receiving measurements comprising at least a filtering pressure difference and/or a pressure difference over the water treatment sub-system (stage 210), determining, based on the measurements, a type and extent of membrane degradation of the water treatment sub-system (stage 220), e.g., degradation due to biological fouling and/or mineral scaling, and adjusting, based on the determined type and extent of the degradation, a UV dose applied to the delivered water to treat biological fouling and/or addition of chemicals to treat mineral scaling in the delivered water (stage 230)—to maximize the performance of the water treatment sub-system (stage 240). Maximization of the efficiency and/or optimization of multiple parameters of the water treatment sub-system (e.g., increasing throughput while reducing maintenance in an RO facility) may be carried out by machine learning and/or artificial intelligence algorithms, as described above.

In various embodiments, method 200 may further comprise receiving additional measurements of at least one of: throughput, pressure, temperature and/or quality of product water and brine from the water treatment sub-system, UV transmittance and/or illumination intensity of the UV treatment module, and/or amounts and types of added chemicals, and applying ML algorithms to analyze the measurements over time and further adjusting the UV dose and the addition of chemicals to maximize the performance (stage 235).

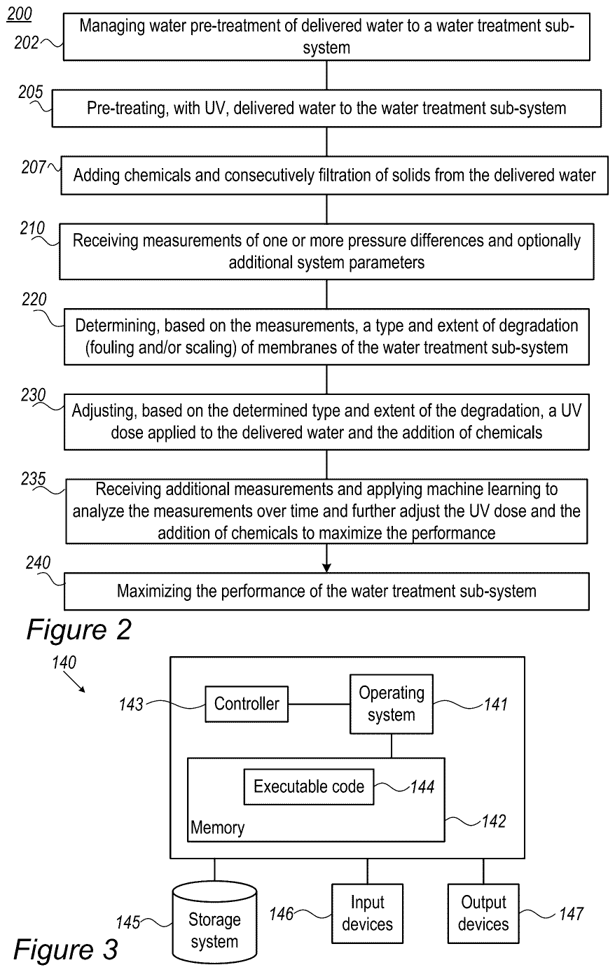

FIG. 3 is a high-level block diagram of exemplary processing unit(s) 140, which may be used with embodiments of the present invention. Processing unit(s) 140 may include one or more controller or processor 143 that may be or include, for example, one or more central processing unit processor(s) (CPU), one or more Graphics Processing Unit(s) (GPU or general-purpose GPU—GPGPU), a chip or any suitable computing or computational device, an operating system 141, a memory 142, a storage 145, input devices 146 and output devices 147.

Operating system 141 may be or may include any code segment designed and/or configured to perform tasks involving coordination, scheduling, arbitration, supervising, controlling, or otherwise managing operation of processing unit(s) 140, for example, scheduling execution of programs. Memory 142 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory 142 may be or may include a plurality of possibly different memory units. Memory 142 may store for example, instructions to carry out a method (e.g., code 144), and/or data such as user responses, interruptions, etc.

Executable code 144 may be any executable code, e.g., an application, a program, a process, task or script. Executable code 144 may be executed by controller 143 possibly under control of operating system 141. For example, executable code 144 may when executed cause the production or compilation of computer code, or application execution such as VR execution or inference, according to embodiments of the present invention. Executable code 144 may be code produced by methods described herein. For the various modules and functions described herein, one or more computing devices and/or components of processing unit(s) 140 may be used. Devices that include components similar or different to those included in processing unit(s) 140 may be used and may be connected to a network and used as a system. One or more processor(s) 143 may be configured to carry out embodiments of the present invention by for example executing software or code.

Storage 145 may be or may include, for example, a hard disk drive, a floppy disk drive, a Compact Disk (CD) drive, a CD-Recordable (CD-R) drive, a universal serial bus (USB) device or other suitable removable and/or fixed storage unit. Data such as instructions, code, VR model data, parameters, etc. may be stored in a storage 145 and may be loaded from storage 145 into a memory 142 where it may be processed by controller 143. In some embodiments, some of the components shown in FIG. 3 may be omitted.

Input devices 146 may be or may include for example a mouse, a keyboard, a touch screen or pad or any suitable input device. It will be recognized that any suitable number of input devices may be operatively connected to processing unit(s) 140 as shown by block 146. Output devices 147 may include one or more displays, speakers and/or any other suitable output devices. It will be recognized that any suitable number of output devices may be operatively connected to processing unit(s) 140 as shown by block 147. Any applicable input/output (I/O) devices may be connected to processing unit(s) 140, for example, a wired or wireless network interface card (NIC), a modem, printer or facsimile machine, a universal serial bus (USB) device or external hard drive may be included in input devices 146 and/or output devices 147.

Embodiments of the invention may include one or more article(s) (e.g., memory 142 or storage 145) such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein.

Certain embodiments comprise a computer program product (e.g., being part of and/or implemented by processing unit(s) 140) comprising a non-transitory computer readable storage medium having computer readable program embodied therewith, the computer readable program comprising computer readable program configured to implement on or more parts of methods 200, e.g., comprise computer readable program configured to receive measurements comprising at least a filtration pressure difference and/or a pressure difference over membranes of the water treatment sub-system, computer readable program configured to determine, based on the measurements, a type and extent of degradation of the membranes of the water treatment sub-system due to biological fouling and/or mineral scaling, and/or computer readable program configured to adjust, based on the determined type and extent of the degradation, a UV dose applied to the delivered water to treat biological fouling and/or addition of chemicals to the delivered water to treat mineral scaling—to maximize a performance of the water treatment sub-system.

In various embodiments, the computer readable program may further comprise computer readable program configured to receive additional measurements of at least one of: throughput, pressure, temperature and/or quality of product water and brine from the water treatment sub-system, UV transmittance and/or illumination intensity of the UV treatment module, and/or amounts and types of added chemicals, and computer readable program configured to apply ML algorithms to analyze the measurements over time and further adjusting the UV dose and the addition of chemicals to maximize the performance.

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram or portions thereof.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams may represent a module, segment, or portion of code, which comprises one or more executable instructions for implementing the specified logical function(s). It should also be noted that, in some alternative implementations, the functions noted in the portion may occur out of the order noted in the figures. For example, two portions shown in succession may, in fact, be executed substantially concurrently, or the portions may sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

Embodiments of the present invention provide efficient and economical methods and mechanisms for removing color from polylactic acid (PLA), e.g., using UV, and thereby provide improvements to various technological fields that utilize PLA. Lactic acid obtained, e.g., from biological reactors usually requires a clarification step in order to be used in a wide range of applications. The inventors have found out that using MPUV would result in clarification of the lactic acid or lactate solution with no influence on their characteristics and usability to produce PLA. Specifically, applying UV from MPUV lamps on the solution from the biological reactor (which includes lactic acid and/or lactate) was found to remove contaminants and impurities that caused yellow color of the solution.

Purification systems and methods are provided for purifying solutions of lactic acid and/or lactate by UV radiation, to improve the quality of polylactic acid (PLA) produced therefrom. The color of the solution is monitored to tune the wavelength and intensity of the applied UV.

For example, MPUV in the wavelength range of 200-400 nm may be used to remove color and purify solutions with lactic acid or lactate. The UV dose for the color removal when applied to lactic acid solutions may be in the range of 850-2000 mJ/cm², or within subranges thereof; and the UV dose for the color removal when applied to lactate solutions may be in the range 500-2000 mJ/cm², or within subranges thereof. Using an online sensor for color detection may result in energy optimization to monitor and adjust lamp power wavelength and/or output, as the reaction occurs with first order kinetics with higher efficiency as the reaction progresses.

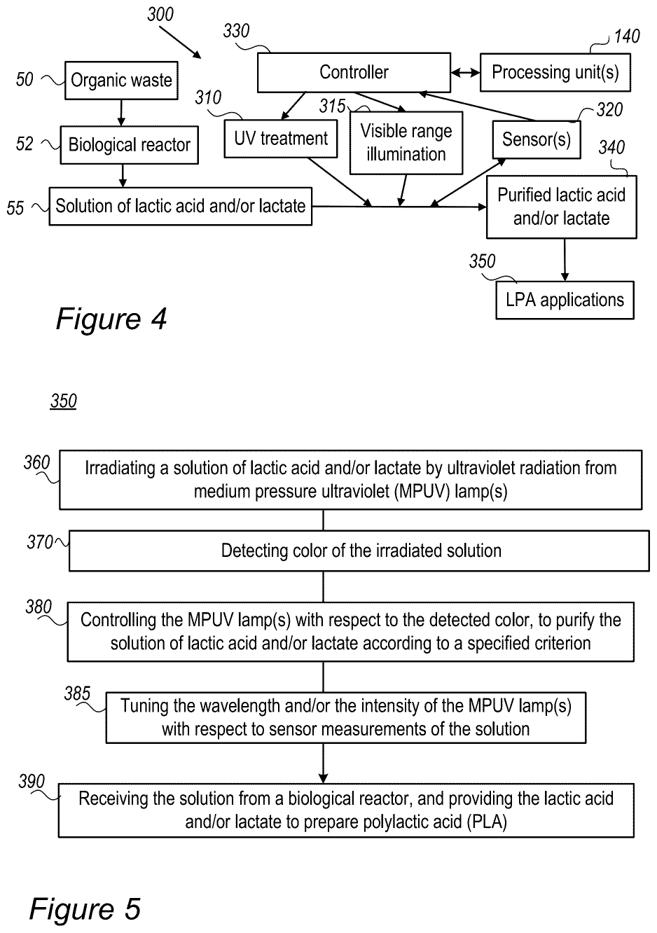

FIG. 4 is a high-level schematic block diagram of a purification system 300 for purifying a solution of lactic acid and/or lactate 55 received from a biological reactor 52, according to some embodiments of the invention. Typically, organic waste 50 is converted in biological reactor 52 to yield solution 55, which requires further purification, especially for high end use of LPA produced from the lactic acid and/or lactate. Accordingly, purification system 300 may be applied to solutions that include lactic acid and/or lactate received from a biological reactor, and used after purification to prepare polylactic acid (PLA).

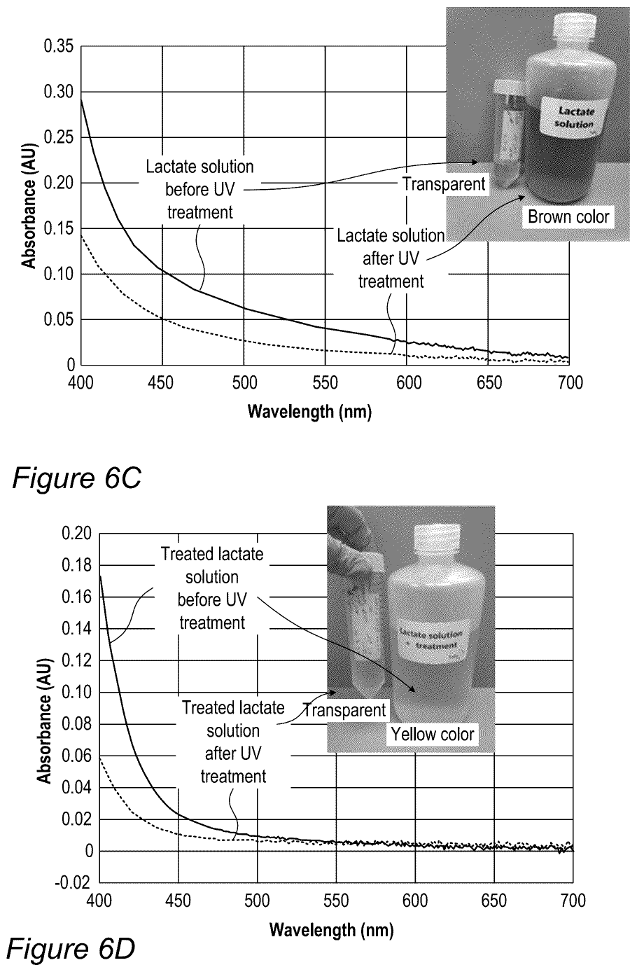

Purification system 300 comprises at least one medium pressure ultraviolet (MPUV) lamp 310 configured to irradiate solution 55 of lactic acid and/or lactate (illustrated schematically), at least one sensor 320 for color detection of the irradiated solution, and a controller 330 configured to control the UV treatment by MPUV lamp(s) 310 with respect to measurements of sensor(s) 320, to purify the solution of lactic acid and/or lactate according to a specified criterion—to yield purified lactic acid and/or lactate 340 (e.g., in solution) for various LPA applications 350. In certain embodiments, sensor(s) 320 may carry out color measurement within the visible range (400-700 nm) and/or within the UV range (200-400 nm). Certain embodiments further comprise an illumination source for irradiation in the visible range between 400-700 nm, configured to illuminate solution 55 for measurement purposes and/or possibly to further purify solution 55. Examples for purification criteria may comprise optical criteria such as maximal absorption or optical density (OD) threshold(s) at one or more wavelengths or wavelength ranges (see, e.g., FIGS. 6A-6D).

In various embodiments, MPUV lamp(s) may be configured to irradiate solution 55 with ultraviolet radiation in the range of 200-400 nm, and the applied UV dose may range between 850-2000 mJ/cm² (or subranges thereof) when applied to lactic acid solutions and ranges between 500-2000 mJ/cm² (or subranges thereof) when applied to lactate solutions.

Controller 330 may be configured to tune the wavelength and/or the intensity of MPUV lamp(s) 310 with respect to measurements by sensor(s) 320. The UV lamp(s) in UV treatment unit 310 may be tuned to optimize color removal from solution 55 of lactic acid and/or lactate received from biological reactor 52.

In some embodiments, one or more of the MPUV lamp(s) may be configured to emit UV light having wavelength(s) between 260-400 nm or possibly between 260-300 nm. The pressure of the lamp may be greater than 1.6 bar, 3 bar or possible greater than 5, 6 or 7 bar. The lamp may be composed of PS (pure silica synthetic quartz). In some embodiments, one or more of the MPUV lamp(s) may be configured to emit UV light having wavelength(s) between 200 and 245 nm, possibly within a wavelength range of between 200 and 220 nm. One or more of the MPUV lamp(s) may be monochromatic, emitting UV light having a single wavelength in the range of 200-245 nm, or possibly in the range of 200-220 nm. Polychromatic and/or monochromatic UV lamp(s) 310 in both the upper and lower wavelength ranges may be customized to purify lactic acid and/or lactate solution 55, e.g., customized with respect to the response sensitivity of the solution of lactic acid and/or lactate received from the biological reactor. Based on the response sensitivity of the solution, higher pressure optimized MP lamps may contribute more to a cumulative intensity than the lower pressure MP lamp. The pressure of lamps(s) may be adjusted to match the spectral response of the solution to UV light.

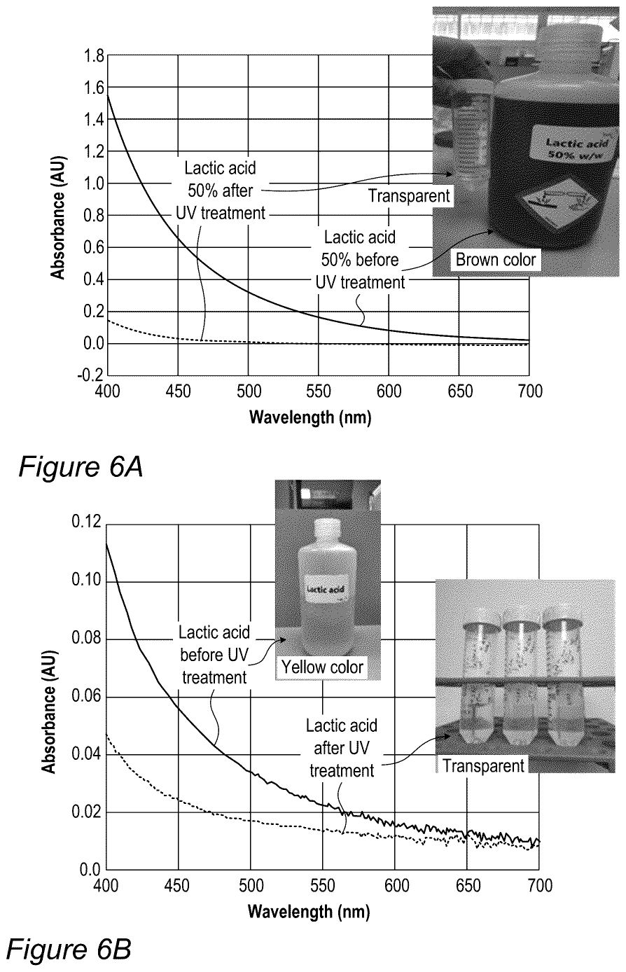

FIG. 5 is a high-level flowchart illustrating purification methods 350, according to some embodiments of the invention. The method stages may be carried out with respect to system 300 and/or controller 330 described above, which may optionally be configured to implement methods 350. Methods 350 may be at least partially implemented by at least one computer processor, e.g., in controller 330 associated with processing unit(s) 140 disclosed herein. Certain embodiments comprise computer program products comprising a computer readable storage medium having computer readable program embodied therewith and configured to carry out the relevant stages of methods 350. Methods 350 may comprise the following stages, irrespective of their order.

Purification method 350 may comprise irradiating a solution of lactic acid and/or lactate by ultraviolet radiation from at least one medium pressure ultraviolet (MPUV) lamp (stage 360), detecting color of the irradiated solution, e.g., using sensor measurements in the visible range (400-700 nm) and/or in the UV range (200-400 nm) (stage 370), and controlling the at least one MPUV lamp with respect to the detected color, to purify the solution of lactic acid and/or lactate according to a specified criterion (stage 380).

Irradiating the solution (stage 360) may be carried out with an applied UV dose that ranges between 850-2000 mJ/cm² when applied to lactic acid solutions and ranges between 500-2000 mJ/cm² when applied to lactate solutions.

In certain embodiments, purification method 350 may comprise tuning the wavelength of the at least one MPUV lamp with respect to measurements by the at least one sensor (stage 385).

Purification method 350 may further comprise receiving the solution from a biological reactor, and providing the lactic acid and/or lactate to prepare polylactic acid (PLA) (stage 390).

Certain embodiments comprise computer program product comprising a non-transitory computer readable storage medium having computer readable program embodied therewith, the computer readable program configured to control at least one medium pressure ultraviolet (MPUV) lamp irradiating a solution of lactic acid and/or lactate by ultraviolet radiation for purification, with respect to a detected color of the irradiated solution, according to a specified criterion. The computer program product may further comprise computer readable program configured to tune the wavelength of the at least one MPUV lamp with respect to the detected color.

FIGS. 6A-6D provide non-limiting examples for UV treatment of solutions 55, according to some embodiments of the invention. The figures provide visible light absorbance curves for a 50% lactic acid solution (FIG. 6A), pure lactic acid (FIG. 6B), a lactate solution (FIG. 6C), and lactate solution treated by conventional means (FIG. 6D)—each before and after UV treatment, and with images illustrating the purification of the respective solutions, which become transparent after UV treatment.

In purification system 300, sensor(s) 320 may be configured to measure specific wavelength ranges (e.g., within the visible range, e.g., between 400-500 nm, 400-600 nm, a broader range of the visible, or subranges or values therein, and/or within the UV range below 400 nm, or around 400 nm, within 400-450 nm, 350-450 nm, etc.) to detect the color of the solution (in a broad sense-visible light color and/or UV color), and controller 330 may be configured to adjust accordingly the wavelength and/or intensity of applied UV by treatment unit 310 (e.g., by controlling which of several UV lamps is operated and at what intensity, possibly also irradiating the solution with visible light).

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A water treatment system for pre-treating water delivered to a water treatment sub-system, the water treatment system comprising:
a ultraviolet (UV) treatment module configured to treat the delivered water with UV,
a chemical treatment module comprising a chemical addition unit configured to add chemicals to the delivered water and a filtration unit configured to filter solids from the delivered water, and
the monitoring module of claim 4.

2. The water treatment system of claim 1, wherein the water treatment sub-system is a reverse osmosis (RO) desalination facility.

3. The water treatment system of claim 1, wherein:
the monitoring module is further configured to further receive measurements of at least one of: throughput, pressure, temperature and/or quality of product water and brine from the water treatment sub-system, UV transmittance and/or illumination intensity of the UV treatment module, and/or amounts and types of added chemicals, and
the monitoring module comprises or is associated with an analysis unit configured to apply machine learning (ML) algorithms to analyze the measurements over time and further adjust the UV dose and the addition of chemicals to maximize the performance.

4. A monitoring module in a water treatment system for pre-treating water delivered to a water treatment sub-system, the monitoring module configured to:
receive measurements comprising at least a pressure difference over a filtration unit of the water treatment system and/or a pressure difference over the water treatment sub-system,
determine, based on the measurements, a type and extent of degradation of membranes of the water treatment sub-system due to biological fouling and/or mineral scaling, and
adjust, based on the determined type and extent of the degradation, a UV dose applied by a UV treatment module to the delivered water to treat biological fouling and/or an addition of chemicals to the delivered water to treat mineral scaling—to maximize a performance of the water treatment sub-system.

5. The monitoring module of claim 4, wherein the water treatment sub-system is a reverse osmosis (RO) desalination facility.

6. The monitoring module of claim 4, further configured to further receive measurements of at least one of: throughput, pressure, temperature and/or quality of product water and brine from the water treatment sub-system, UV transmittance and/or illumination intensity of the UV treatment module, and/or amounts and types of added chemicals, wherein the monitoring module comprises or is associated with an analysis unit configured to apply ML algorithms to analyze the measurements over time and further adjust the UV dose and the addition of chemicals to maximize the performance.

7. A method of managing water pre-treatment of delivered water to a water treatment sub-system, the method comprising:
receiving measurements comprising at least a filtering pressure difference and/or a pressure difference over the water treatment sub-system,
determining, based on the measurements, a type and extent of degradation of membranes of the water treatment sub-system due to biological fouling and/or mineral scaling, and
adjusting, based on the determined type and extent of the degradation, a UV dose applied to the delivered water to treat biological fouling and/or addition of chemicals to the delivered water to treat mineral scaling—to maximize a performance of the water treatment sub-system.

8. The method of claim 7, wherein the water treatment sub-system is a reverse osmosis (RO) desalination facility.

9. The method of claim 7, further comprising:
receiving additional measurements of at least one of: throughput, pressure, temperature and/or quality of product water and brine from the water treatment sub-system, UV transmittance and/or illumination intensity of the UV treatment module, and/or amounts and types of added chemicals, and
applying ML algorithms to analyze the measurements over time and further adjusting the UV dose and the addition of chemicals to maximize the performance.

10. A water pre-treatment method comprising:
treating, with UV, delivered water to a water treatment sub-system,
adding chemicals and consecutively filtering the delivered water, and
managing water pre-treatment of delivered water to a water treatment sub-system according to the method of claim 7.

11. The water pre-treatment method of claim 10, wherein the water treatment sub-system is a reverse osmosis (RO) desalination facility.

12. The water pre-treatment method of claim 10, further comprising:
receiving additional measurements of at least one of: throughput, pressure, temperature and/or quality of product water and brine from the water treatment sub-system, UV transmittance and/or illumination intensity of the UV treatment module, and/or amounts and types of added chemicals, and
applying ML algorithms to analyze the measurements over time and further adjusting the UV dose and the addition of chemicals to maximize the performance.

13. A computer program product comprising a non-transitory computer readable storage medium having computer readable program embodied therewith, the computer readable program comprising:
computer readable program configured to receive measurements comprising at least a filtering pressure difference and/or a pressure difference over the water treatment sub-system,
computer readable program configured to determine, based on the measurements, a type and extent of degradation of membranes of the water treatment sub-system due to biological fouling and/or mineral scaling, and
computer readable program configured to adjust, based on the determined type and extent of the degradation, a UV dose applied to the delivered water to treat biological fouling and/or addition of chemicals to the delivered water to treat mineral scaling—to maximize a performance of the water treatment sub-system.

14. The computer program product of claim 13, wherein the water treatment sub-system is a reverse osmosis (RO) desalination facility.

15. The computer program product of claim 13, further comprising:
computer readable program configured to receive additional measurements of at least one of: throughput, pressure, temperature and/or quality of product water and brine from the water treatment sub-system, UV transmittance and/or illumination intensity of the UV treatment module, and/or amounts and types of added chemicals, and
computer readable program configured to apply ML algorithms to analyze the measurements over time and further adjusting the UV dose and the addition of chemicals to maximize the performance.

16-27. (canceled)
